(19) United States
(12) Patent Application Publication
(10) Pub. No.: US 2016/0106813 A1
(43) Pub. Date: Apr. 21, 2016

(54) CGRP RECEPTOR AGONIST FOR HIV TREATMENT OR PREVENTION

(71) Applicants: CENTRE NATIONAL DE LA RECHERCHE SCHIENTIFIQUE (CNRS), Paris (FR); INSTITUT NATIONAL DE LA SANTE ET DE LA RECHERCHE MEDICALE (INSERM), Paris (FR); UNIVERSITE PARIS DESCARTES, Paris (FR)

(72) Inventors: Morgane BOMSEL, Paris (FR); Yonatan GANOR, Paris (FR)

(21) Appl. No.: 14/889,762

(22) PCT Filed: Mar. 28, 2014
(86) PCT No.: PCT/EP2014/056356

§ 371 Date: Nov. 6, 2015

(30) Foreign Application Priority Data
Mar. 29, 2013 (EP) 13305417.1

(51) Int. Cl. A61K 38/23; G01N 33/50

(52) U.S. Cl. CPC A61K 38/23; G01N 33/5047; G01N 2333/16

(57) ABSTRACT

A first object of the invention is CGRP or an agonist of the CGRP receptor for use in the prevention of an HIV infection in a human subject. A second object of the invention is a method for selecting an active compound for the prevention of an HIV infection in a human subject, comprising the steps of: a) contacting Langerhans cells with a candidate compound; b) in vitro infecting of Langerhans cells from step a) by at least one HIV variant, c) measuring of at least one of the following parameters: HIV content, adhesive potential of Langerhans cells, in particular to TCs, secretion of anti-HIV chemokines by Langerhans cells. A third object of the invention is therefore a composition comprising CGRP or agonist of the CGRP receptor for use in the prevention of an HIV infection in a human subject. A fourth object of the invention is a method for the in vitro diagnosis of an HIV infection in a human subject, characterized in that it comprises determining the level of CGRP from a biological sample from said subject. A fifth object of the invention is a method for assaying the efficiency of HAART in the treatment of HIV infection in a subject, characterized in that it comprises determining the level of CGRP from a biological sample from said subject.

Human immunodeficiency virus (HIV) is a lentivirus (a member of the retrovirus family) that causes acquired immunodeficiency syndrome (AIDS), a condition in humans in which progressive failure of the immune system allows life-threatening opportunistic infections and cancers to thrive.

HIV infects vital cells in the human immune system such as helper T-cells (specifically CD4+ T-cells), macrophages, and dendritic cells. HIV infection leads to low levels of CD4+ T-cells through three main mechanisms: first, direct viral killing of infected cells; second, increased rates of apoptosis in infected cells; and third, killing of infected CD4+ T-cells by CD8 cytotoxic lymphocytes that recognize infected cells. When CD4+ T-cell numbers decline below a critical level, cell-mediated immunity is lost, and the body becomes progressively more susceptible to opportunistic infections.

Due to the great variability in the HIV genome, which results from mutation, recombination, insertion and/or deletion, HIV has been classified in groups, subgroups, types, subtypes and genotypes. Two groups of HIV have been characterized: HIV-1 and HIV-2. HIV-1 is the virus that was initially discovered and termed both LAV and HTLV-III. It is more virulent, more infectious than HIV-2, and is the cause of the majority of HIV infections globally. The lower infectivity of HIV-2 compared to HIV-1 implies that fewer of those exposed to HIV-2 will be infected per exposure. Because HIV-2 is less infectious than HIV-1, it remains mostly confined to West Africa. Both groups comprise many subgroups because the HIV genome mutates constantly. The major difference between the groups and subgroups is associated with the viral envelope. HIV-1 is classified into a main subgroup (M), said subgroup M being divided into nine subtypes (clades or subtypes) designed A through J (Hu et al, JAMA 1996, 275:210-216; Korber et al, Science 1998, 280:1868-1871), and a 10th outlier subgroup (O). Many other subgroups resulting from in vivo recombinations of the previous ones also exist (Papathanasopoulos et al, Virus Genes 2003, 26:151-163).

HIV variants may further be classified according to their tropism, i.e. the cell type preferentially infected by said variant. HIV can infect a variety of cells such as CD4+ helper T-cells and macrophages that express the CD4 molecule on their surface. HIV entry into CD4+ T-cells is mediated not only through interaction of the virion envelope glycoproteins (gp120) with the CD4 molecule on the target cells but also with its chemokine co-receptors. HIV variants that use the beta-chemokine receptor CCR5 for entry into CD4+ T-cells are said to have a R5 tropism. It has been found that the CCR5 co-receptor is used by almost all primary HIV-1 isolates regardless of viral genetic subtype. On the other hand, HIV variants that use the alpha-chemokine receptor CXCR4 for entry into CD4+ T-cells are said to have a X4 tropism. HIV variants that use both CCR5 and CXCR4 co-receptors for entry into CD4+ T-cells are said to have a X4R5 tropism.

No curative therapies are actually available for HIV infection, and the treatments administered only aim at slowing the infection process. Moreover, no available therapy has clearly been proven to be able to prevent HIV infection. There is thus still a need for treatments to prevent HIV infection in humans.

Highly Active Antiretroviral Therapy (HAART) is available commercially for HIV-1-infected individuals. Highly Active Antiretroviral Therapy targets various steps of HIV life cycle in susceptible target cells and suppresses viral load to levels below detection. New data suggests the use of Highly Active Antiretroviral Therapy as a prophylactic measure aimed at preventing HIV transmission. Such suggestion is based on recent clinical trials: the CAPRISA-004 study demonstrated that topical administration of a gel containing the antiretroviral agent tenofovir decreased the risk of HIV acquisition among at-risk heterosexual women (Abdool Karim et al, Science 2010, 329:1168-1174). Subsequently, the iPrEx study reported that prophylactic use of a daily oral tablet containing tenofovir and emtricitabine reduced the risk of HIV acquisition among high-risk men who have sex with men (Grant et al, N Engl J Med 2010, 363:2587-2599).

Although effective, Highly Active Antiretroviral Therapy has however undesirable side effects. First, the mandatory strict adherence to Highly Active Antiretroviral Therapy regimens is not always met. Second, long term Highly Active Antiretroviral Therapy often results in development of drug resistant HIV strains.

Hence, new active compounds are required for better prevention of HIV infection.

The invention provides new compounds for use in preventive treatments of an HIV infection in a human subject. The Inventors have surprisingly found that Calcitonin Gene-Related Peptide (CGRP) is able to inhibit HIV transmission and spread from Langerhans cells (LCs) to T-cells (TCs), and in turn the infection of TCs.

CGRP is a sensory neuropeptide that is a member of the calcitonin family of peptides, which includes also calcitonin, adrenomedullin (AM) and amylin (AMY). In humans, CGRP exists in two forms, α-CGRP and β-CGRP. CGRP is produced by alternative splicing of the calcitonin genes in both peripheral and central neurons. These CGRP containing neurons are polymodal nociceptors activated by chemical/thermal/mechanical stimuli, which project to different areas of the brain to transmit these pain inputs to the cortex. CGRP is also released antidromically from peripheral terminals, and contributes to neurogenic inflammation by inducing potent vasodilatation. The plasma levels of CGRP serve as a clinical peripheral marker for the involvement and deregulation of CGRP in several pathological conditions (e.g. are increased in patients with migraine (Villalon and Olesen, Pharmacol Ther 2009, 124:309-323) and decreased in patients with hypertension (Smillie and Brain, Neuropeptides 2011. 45:93-104)). The functional CGRP receptor is an assembly of the seven transmembrane domain G-protein coupled receptor calcitonin receptor-like receptor (CRLR) and an associated single transmembrane domain protein termed receptor activity modifying protein 1 (RAMP1) (Hay D L et al, Pharmacol Rev 2008, 60:143-145), which was previously termed CGRP receptor of type 1. Co-expression of CRLR with RAMP2 and RAMP3 yields receptors that preferentially bind AM (AM₁ and AM₂ receptors, respectively); co-expression of the calcitonin receptor with RAMP1-3 yields receptors that preferentially bind AMY (AMY₁₋₃ receptors, respectively). Although the AM₂, AMY₁ and AMY₃ receptors are also activated by CGRP, they are only weakly antagonized by the truncated CGRP receptor antagonist CGRP₈₋₃₇ and were hence previously termed CGRP receptors of type 2 (Hay, Clin Exp Pharmacol Physiol, 34:963-971, 2007). However, this historical classification is obsolete, and it is currently accepted that the CGRP receptor corresponds to the CRLR/RAMP1 complex and should be termed “CGRP receptor” (Hay et al, Pharmacol Rev 2008, 60:143-145).

Moreover, the Inventors have established that the therapeutic effect of CGRP in the prevention of an HIV infection is mediated through activation of the CGRP receptor. In contrast, activation of the related AMY₁ receptor that is also activated by CGRP, is therapeutically ineffective.

The invention is therefore particularly unexpected, as the use of CGRP, nor of agonists of the CGRP receptor, has so far never been considered as a possible anti-HIV clinical strategy.

A first object of the invention is CGRP or an agonist of the CGRP receptor for use in the prevention of an HIV infection in a human subject.

Thus, the invention also has for object the use of CGRP or agonist of the CGRP receptor for the manufacture of a medicament intended for the prevention of an HIV infection in a human subject.

In addition, the invention provides for a method for the prevention of an HIV infection in a human subject in need thereof, comprising administering an effective amount of CGRP or of an agonist of the CGRP receptor to said subject.

Moreover, the Inventors have characterized the biological pathway associated with the therapeutic effect of CGRP and have set up a method for selecting active compounds that are appropriate for the prevention of an HIV infection.

A second object of the invention is a method for selecting an active compound for the prevention of an HIV infection in a human subject, comprising the steps of:
a) contacting Langerhans cells with a candidate compound;
b) in vitro infecting of Langerhans cells from step a) by at least one HIV variant,
c) measuring of at least one of the following parameters:
   HIV content,
   adhesive potential of Langerhans cells, in particular to TCs,
   secretion of anti-HIV chemokines by Langerhans cells.

The compounds of the invention may be formulated into composition in order to facilitate their administration.

A third object of the invention is therefore a composition comprising CGRP or agonist of the CGRP receptor for use in the prevention of an HIV infection in a human subject.

The Inventors have additionally discovered that the level of CGRP in biological samples from human subjects can be used as a biomarker to efficiently diagnose HIV infections. A fourth object of the invention is a method for the in vitro diagnosis of an HIV infection in a human subject, characterized in that it comprises determining the level of CGRP from a biological sample from said subject.

A fifth object of the invention is a method for assaying the efficiency of HAART in the treatment of HIV infection in a subject, characterized in that it comprises determining the level of CGRP from a biological sample from said subject.

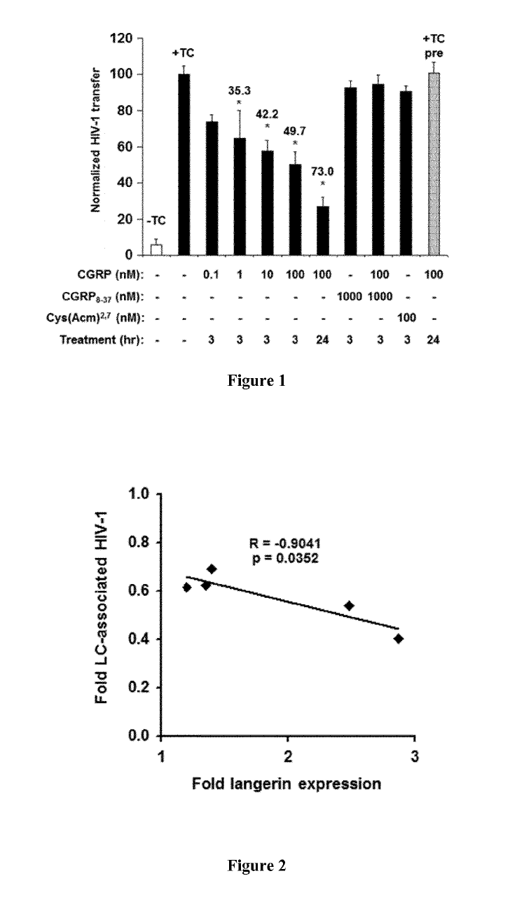

FIG. 1: CGRP inhibits HIV-1 transfer from Langerhans cells to TCs. Monocyte-derived Langerhans cells (MDLCs, black bars) or CD4+ TCs (+TC pre, grey bar) were pretreated, as indicated, for 3-24 h with 0.1-100 nM CGRP; 1000 nM CGRP₈₋₃₇ (added alone or 10 min before addition of CGRP); or 100 nM Cys(Acm)2,7. Untreated Langerhans cells alone (−TC, white bars) served as background and with TCs (+TC) as the 100% set point. Results represent mean±s.e.m. normalized HIV-1 transfer derived from n=7 experiments; numbers represent % inhibition. *p=0.0292, 0.0006, <0.0001, <0.0001 for 1 nM/3 h, 10 nM/3 h, 100 nM/3 h, 100 nM/24 h CGRP vs. +TC, respectively.

FIG. 2: langerin expression and HIV-1 degradation in vitro. MDLCs were pretreated with CGRP (100 nM, 24 h), pulsed with HIV-1, lysed, and HIV-1 measured in the cell lysates. Langerin surface levels were measured following CGRP-pretreatment and before HIV-1 pulsing. Results are presented as a linear correlation between fold langerin expression vs. fold HIV-1 content in the cell lysates ([CGRP-treated/untreated]), and each point denotes a different donor.

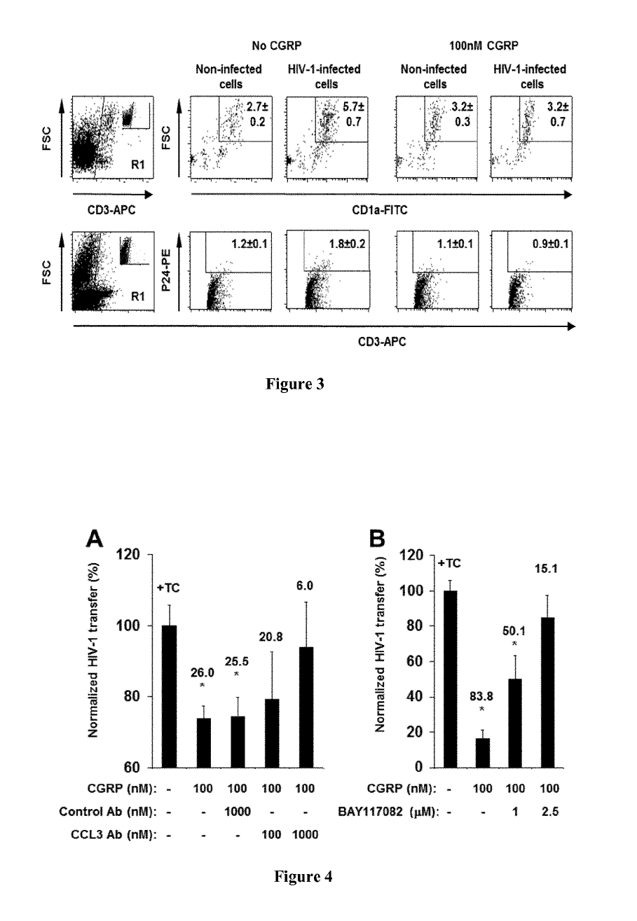

FIG. 3: LC-TC conjugate formation and mucosal TC infection. Inner foreskin explants were either left untreated (no CGRP) or CGRP-pretreated (100 nM CGRP) for 24 h, exposed for 4 h to either non-infected or HIV-1-infected cells, processed for preparation of epidermal/dermal single-cell suspensions, and evaluated by flow cytometry. Shown are representative FACS profiles with numbers representing the mean±s.e.m. (n=3) percentages within the entire cell-suspensions of either CD3+/CD1a+/high FSC LC-T-cell conjugates (top, epidermis) or CD3+/p24+ cells (bottom, dermis). Cells were first gated on CD3+ cells (R1 gates, left profiles, inserts shows staining with matched isotype controls).

FIG. 4: Effect of CGRP on CCL3/MIP-1α secretion. HIV-1 transfer experiments in the presence of either control Ab, neutralizing Ab to CCL3 (A; MUTZ3-derived Langerhans cells) or the NFκB inhibitor BAY117082 (B; MDLCs). Results (n=4) are presented as in (A). *p=0.0008 and 0.0307 for CGRP alone or CGRP+control Ab vs. +TC, respectively (A); *p=0.0014 and 0.0058 for CGRP alone or in the presence of 1 μM BAY117082 vs. +TC, respectively (B).

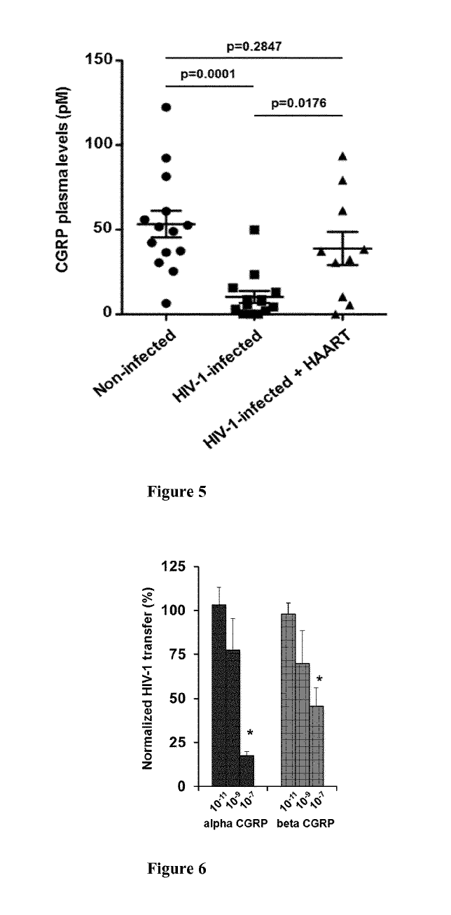

FIG. 5: CGRP plasma levels as a biomarker to diagnose HIV infections and the effectiveness of HAART. CGRP levels were evaluated in plasma of 14 treatment-naïve and 10 HAART-treated HIV-1-infected individuals compared to 14 healthy controls. Horizontal lines denote the means. *p=0.0004 ANOVA.

FIG. 6: alpha and beta CGRP inhibit HIV-1 transfer from LCs to T-cells. MDLCs were left untreated or pretreated for 24 h with the indicated molar concentrations of either alpha CGRP (black bars), beta CGRP (dark grey bars). The cells were then pulsed with 10³ TCID50 HIV-1 JRCSF for 2 h, washed, and incubated for a week with autologous CD4+ T-cells. HIV-1 replication was measured in the co-culture supernatants by p24 ELISA. Results represent mean±SEM from n=4 human donors of HIV-1 transfer normalized against untreated cells serving as the 100% set point. * p=0.0017 and p=0.0356 for 10⁻⁷M vs 10⁻¹¹M alpha CGRP and beta CGRP, respectively; Student's t-test.

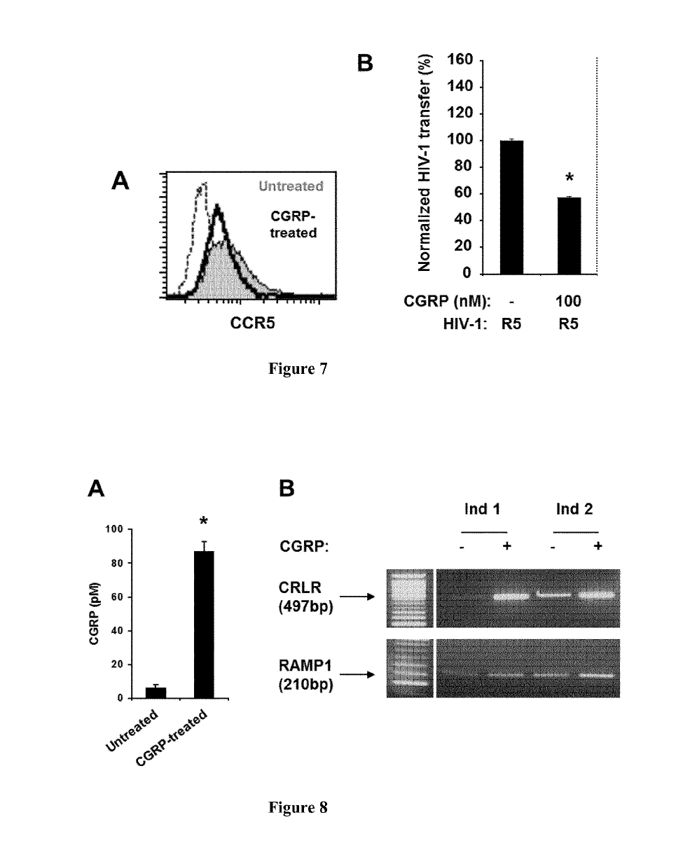

FIG. 7: CGRP decreases CCR5 expression and inhibits HIV-1 R5 transfer. (A) Representative FACS histograms showing surface expression of the HIV-1 co-receptors CCR5 (grey histograms) and CGRP-treated (100 nM, 24 h; black histograms) MDLCs vs matched isotype controls (broken line histograms). (B) MDLCs were left untreated or pretreated with 100 nM CGRP for 24 h. The cells were then pulsed with 10³ TCID50 of either HIV-1 JRCSF (R5) for 2 h, washed, and incubated for a week with autologous CD4+ T-cells. HIV-1 replication was measured in the co-culture supernatants by p24 ELISA. Results represent mean±SEM from n=3 human donors of HIV-1 transfer normalized against untreated cells serving as the 100% set point. * p=0.0012 for CGRP-treated vs untreated cells pulsed with R5 HIV-1; Student's t-test.

FIG. 8: Expression of CGRP itself and its cognate receptor in LCs are up-regulated by CGRP. (A) MDLCs were left untreated or pretreated with 100 nM CGRP for 24 h. The cells were then washed extensively and cultured in fresh medium for additional 24 h. The levels of CGRP secreted to the culture supernatants were measured by CGRP ELISA. Results represent mean±SEM from n=2 human donors. * p=0.0061 for CGRP-treated vs untreated; Student's t-test. (B) RT-PCR images for CRLR and RAMP1 in MDLCs from n=2 human donors, either left untreated or pretreated with 100 nM CGRP for 24 h.

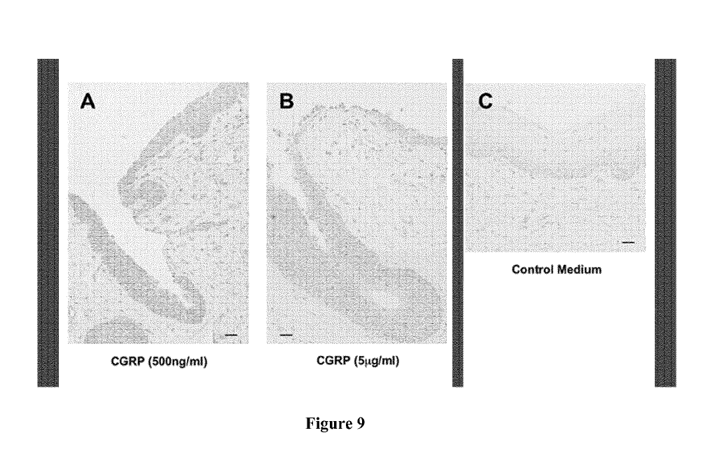

FIG. 9: Mucosally applied CGRP penetrates the epithelial compartment of penile tissue. Whole penile tissue was obtained from a healthy individual undergoing elective gender reassignment, and polarized explants were prepared from the fossa navicularis region. The explants were exposed in a polarized manner to biotinylated CGRP at 500 nM (A) or 5 μM (B), as well as to control medium RPMI (C) as negative control for 3 h at 37° C. Next the explants were extensively washed, fixed in 4% PFA for 48 h, embedded in paraffin, and serial 4 μm section were cut. Following deparaffinization and antigen retrieval, the sections were incubated with a pre-diluted solution of streptavidin-HRP for 30 min at room temperature. Following additional washings, the HRP staining was visualized using AEC peroxidase substrate, and section were counterstained with hematoxylin; Scale bars=10 μm.

A first object of the invention is CGRP or an agonist of the CGRP receptor for use in the prevention of an HIV infection in a human subject.

Thus, the invention also has for object the use of CGRP or an agonist of the CGRP receptor for the manufacture of a medicament intended for the prevention of an HIV infection in a human subject.

In addition, the invention provides for a method for the prevention of an HIV infection in a human subject in need thereof, comprising administering an effective amount of CGRP or of an agonist of the CGRP receptor, to said subject.

By “Calcitonin Gene-Related Peptide” and “CGRP”, it is herein referred to any of the human peptides α-CGRP, β-CGRP, their functional variants and fragments. Preferably, in the context of the invention, CGRP is the human peptide α-CGRP of sequence the sequence SEQ ID no.1 or the human peptide β-CGRP of sequence the sequence SEQ ID no.2. Yet preferably, CGRP is the human peptide α-CGRP of sequence the sequence SEQ ID no.1.

The human peptide α-CGRP (UniProtKB/Swiss-Prot ref.: P06881.3) is encoded by the human gene CALCA (NCBI ref: NG_015960.1) and has the sequence SEQ ID no.1:

Ala-Cys-Asp-Thr-Ala-Thr-Cys-Val-Thr-His-Arg-Leu-Ala-Gly-Leu-Leu-Ser-Arg-Ser-Gly-Gly-Val-Val-Lys-Asn-Asn-Phe-Val-Pro-Thr-Asn-Val-Gly-Ser-Lys-Ala-Phe-NH2.

The human peptide β-CGRP (UniProtKB/Swiss-Protref.: P10092.1) is encoded by the human gene CALCB, and has the sequence SEQ ID no.2:

Ala-Cys-Asn-Thr-Ala-Thr-Cys-Val-Thr-His-Arg-Leu-Ala-Gly-Leu-Leu-Ser-Arg-Ser-Gly-Gly-Met-Val-Lys-Ser-Asn-Phe-Val-Pro-Thr-Asn-Val-Gly-Ser-Lys-Ala-Phe-NH2

Preferably, by the terms “Calcitonin Gene-Related Peptide” and “CGRP”, it is herein referred to the human peptide α-CGRP, its functional variants and fragments. According to the invention, the terms “protein”, “peptide” and “polypeptide” are used interchangeably and refer to a polymer of amino acid monomers having no specific length, wherein the amino acid monomers are linked by peptide bonds. Also covered by this definition, are peptides having undergone post-translational modifications such as peptides having covalent attachment of glycosyl groups, acetyl groups, phosphate groups, lipid groups, and the like.

By “functional variant of a specific peptide” that has a biological activity of interest, it is herein referred to peptides which peptide sequence differ from the amino acid sequence of said specific peptide, but that generally retains the biological activity of said specific peptide.

By functional variant of CGRP that has a biological activity of interest, it is herein referred to peptides which peptide sequence differ from the amino acid sequence CGRP, but that generally retains all the biological activity of CGRP.

By functional variant of α-CGRP that has a biological activity of interest, it is herein referred to peptides which peptide sequence differ from the amino acid sequence α-CGRP, but that generally retains all the biological activity of α-CGRP.

By functional variant of β-CGRP that has a biological activity of interest, it is herein referred to peptides which peptide sequence differ from the amino acid sequence β-CGRP, but that generally retains the biological activity of β-CGRP.

According to the invention, the terms “activity”, “function”, “biological activity”, and “biological function” are synonymous for the purpose of the present invention and have to be understood as it is commonly known in the art. Preferably, the biological activity of CGRP in the prevention of an HIV infection in a human subject is that of a ligand that binds to and activates CGRP receptor. Preferably, the biological activity of α-CGRP in the prevention of an HIV infection in a human subject is that of a ligand that binds to and activates CGRP receptor. Preferably, the biological activity of β-CGRP in the prevention of an HIV infection in a human subject is that of a ligand that binds to and activates CGRP receptor.

Activation of the CGRP receptor results in Gas-mediated activation of adenylate cyclase, with a subsequent increase in cAMP and activation of PKA. Such events result in either opening of K+ channels directly in smooth muscle cells (enthodelium-independent mechanism) or NO release from endothelial cells (endothelium-dependent mechanism), both leading to relaxation of smooth muscle cells and vasodilatation. Other known signaling pathways of the CGRP receptor include Gαq/11-mediated PLCβ1 activation and MAPK activation (Walker et al, Trends Pharmacol Sci 2010, 31:476-483). Although CGRP increases cAMP production in Langerhans cells, such increase alone was not responsible for the reported capacity of CGRP to inhibit LC antigen presentation (Asahina et al, PNAS 1995, 92:8323-8327), suggesting that different signaling pathways contribute to CGRP-induced vasodilatation in smooth muscle cells vs. immunosuppression in Langerhans cells. Later studies showed that inhibition of LC antigen presentation involves activation of the NFκB pathway (Ding et al, J Invest Dermatol 2007, 127:2357-2367).

Moreover, the Inventors have characterized the signaling pathway associated with the therapeutic effect of CGRP, and found that CGRP activates NFκB in Langerhans cells. Such activation results in elevated secretion of the anti-HIV chemokine CCL3/MIP-1α by Langerhans cells that plays a role in inhibiting HIV transmission and spread from Langerhans cells to TCs, and thus the infection of TCs.

It will thus be obvious to the skilled person that functional variants of CGRP are ligands binding to and activating the CGRP receptor.

Methods for measuring the binding of a compound to a receptor are well known from the skilled person and include immunohistochemistry, ELISA, western blot analysis, surface plasmon resonance (for example with the BIAcore technology), dual polarisation interferometry, and Microscale Thermophoresis (MST), as well as assays used in high throughput screening (HTS) applications, such as scintillation proximity assay (SPA). These assays may for example be performed using soluble CGRP receptor domains, stabilization of CGRP receptor into a membrane-like environment or direct use of solubilized CGRP receptor. For a thorough description of such assays, the skilled person may refer to Jong et al. (J Chromatogr B Analyt Technol Biomed Life Sci.; 2005; 829(1-2):1-25).

The skilled person may also use cell-based assays, such as reporter assays for G-protein-coupled receptors, which use a cell line suitable for stable expression of CGRP receptor and of a reporter gene. For an example of such assays, the skilled person may refer to Saeki et al. (Anal Biochem.; 2010; 400(2):163-72). To be sure that the compound is able to bind native at the target cell surface, the skilled person may further use flow cytometry analysis (for example by FACS), on cells expressing CGRP receptor. According to the present invention, cells expressing CGRP receptor may be either cells naturally expressing said receptor, such as Langerhans cells, or cells transformed with a vector allowing stable expression of said receptor on the plasma membrane of the chosen cell line.

By “activation of the CGRP receptor”, it his herein referred to the activation of adenylate cyclase, and/or the increase of intracellular cAMP, and/or the activation of PKA, and/or the activation of NFκB.

According to the invention, a variant of CGRP has at least 80, 85, 90, 95, 99% of the biological activity of CGRP. According to the invention, a variant of α-CGRP has at least 80, 85, 90, 95, 99% of the biological activity of α-CGRP. According to the invention, a variant of β-CGRP has at least 80, 85, 90, 95, 99% of the biological activity of β-CGRP.

Methods and kits for measuring an increase in cAMP intracellular level in human cells are well known to the skilled person, and commercially available, such as for example the cAMP-Glo™ Assay (Promega) or the LANCE cAMP Assay (PerkinElmer). Methods and kits for measuring the activation of PKA in human cells are well known from the skilled person, and commercially available, such as for example the Human PKA Activation assay (Cat No: IRAAKT2532, Innovative research). Methods and kits for measuring the activation of NFκB in human cells are well known from the skilled person, and commercially available, such as for example the NFkB EMSA Kit (Caltag).

The Inventors have demonstrated that CGRP binding and activation of the CGRP receptor allows for the prevention of the transfer of HIV variants from Langerhans cells infected with said HIV variant to CD4⁺ TCs. Thus, more preferably, the biological activity of CGRP in the prevention of an HIV infection further comprise the prevention of the transfer of HIV variants from Langerhans cells infected with said HIV variant to CD4⁺ TCs.

The prevention of the transfer of HIV variants from Langerhans cells infected with said HIV variant to CD4⁺ TCs can easily be measured by the person skilled in the art according to the method for selecting an active compound for the prevention of an HIV infection of the invention disclosed hereafter.

The term “functional variants of a specific peptide” encompass peptides which have one or more amino acid mutations and/or deletions compared to said specific peptide and retains the biological activity of said specific peptide in the prevention of an HIV infection in a human subject. Preferably, a functional variant of α-CGRP has at least 85%, at least 90%, at least 91%, at least 92%, at least 93%, at least 94%, at least 95%, at least 96%, at least 96%, at least 97%, at least 98%, at least 99% sequence identity with α-CGRP and retains α-CGRP biological activity in the prevention of an HIV infection in a human subject. Preferably, a functional variant of β-CGRP has at least 85%, at least 90%, at least 91%, at least 92%, at least 93%, at least 94%, at least 95%, at least 96%, at least 96%, at least 97%, at least 98%, at least 99% sequence identity with β-CGRP and retains β-CGRP biological activity in the prevention of an HIV infection in a human subject.

By “functional fragments of a specific peptide that has a biological activity of interest, it is herein referred to peptides which peptide sequence is a part of the peptide sequence of the reference peptide, and that can be of any length, provided the biological activity of peptide of reference is retained by said fragment. Preferably, a functional fragment of α-CGRP retains α-CGRP biological activity in the prevention of an HIV infection in a human subject. Preferably, a functional fragment of β-CGRP retains β-CGRP biological activity in the prevention of an HIV infection in a human subject.

According to the present invention, the “percentage identity” between two sequences of nucleic acids or amino acids means the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of the local homology algorithm of Smith and Waterman (1981) [Ad. App. Math. 2:482], by means of the local homology algorithm of Neddleman and Wunsch (1970) [J. Mol. Biol. 48:443], by means of the similarity search method of Pearson and Lipman (1988) [Proc. Natl. Acad. Sci. USA 85:2444] or by means of computer software using these algorithms (GAP, BESTFIT, FASTA and TFASTA in the Wisconsin Genetics Software Package, Genetics Computer Group, 575 Science Dr., Madison, Wis., or by the comparison software BLAST NR or BLAST P).

The percentage identity between two nucleic acid or amino acid sequences is determined by comparing the two optimally-aligned sequences in which the nucleic acid or amino acid sequence to compare can have additions or deletions compared to the reference sequence for optimal alignment between the two sequences. Percentage identity is calculated by determining the number of positions at which the amino acid nucleotide or residue is identical between the two sequences, preferably between the two complete sequences, dividing the number of identical positions by the total number of positions in the alignment window and multiplying the result by 100 to obtain the percentage identity between the two sequences.

For example, the BLAST program, “BLAST 2 sequences” (Tatusova et al., “Blast 2 sequences—a new tool for comparing protein and nucleotide sequences”, FEMS Microbiol., 1999, Lett. 174:247-250) available on the site http://www.ncbi.nlm.nih.gov/gorf/b12.html, can be used with the default parameters (notably for the parameters “open gap penalty”: 5, and “extension gap penalty”: 2; the selected matrix being for example the “BLOSUM 62” matrix proposed by the program); the percentage identity between the two sequences to compare is calculated directly by the program.

CGRP receptors have been described as heterodimeric molecules formed of the calcitonin receptor-like receptor (CRLR), linked to RAMP1. RAMP1 is a transmembrane domain protein of the RAMP family, which further comprises RAMP2 and RAMP3. Several types of receptors are known that can be activated by CGRP: CGRP receptor (formed of CRLR and of RAMP1), AM₂ receptor (formed of CRLR and of RAMP3), and AMY₁ and AMY₃ receptors (formed of the calcitonin receptor and of RAMP1 and RAMP3, respectively). The CGRP receptors can therefore be distinguished from the AM₂, AMY₁ and AMY₃ receptors by the nature of the transmembrane domain of the RAMP family interacting with CRLR.

By “CGRP receptor”, it is herein referred to a protein receptor comprising the CRLR protein Ref NCBI: NP_005786.1), bound to the protein Receptor Activity Modifying Protein 1 (RAMP1) (Ref NCBI: NP_005846.1). Thus, CGRP receptors do not comprise the CRLR protein bound to RAMP2 or RAMP3.

It is well established that in vivo, CGRP receptors are bound to the G protein Gs, which activates adenylate cyclase, with a subsequent increase in cAMP and activation of PKA. Other known signaling pathways of the CGRP receptor include Gq/11-mediated PLCβ1 activation and MAPK activation (Walker et al, Trends Pharmacol Sci 2010, 31:476-483). It is well known from the skilled person that an agonist of a receptor of a specific ligand is a compound that mimics the action of said specific ligand. More specifically, an agonist of a receptor binds to and activates said receptor. An agonist will obviously trigger the same type of biochemical response as the specific ligand would, and in particular modulate the same secondary messengers.

By “agonist of the CGRP receptor”, it is herein referred to a compound that binds to a CGRP receptor and activates said CGRP receptor.

Preferably, the agonist of the invention is active in the prevention of an HIV infection in a human subject. Said activity can easily be determined as explained above. By “compound”, it is herein referred to a chemical compound or a biochemical compound, such as a peptide. Preferably, the agonist of the invention is a biochemical compound. Yet preferably the agonist of the invention is a peptide.

The Inventors have demonstrated that the CGRP receptor linear analogue Cys(Acm)2,7-h-alpha-CGRP does not prevent HIV infection such as CGRP does. It has been reported that Cys(Acm)2,7-h-alpha-CGRP has no stimulatory action on CGRP receptors. This molecule binds CGRP receptors 100-1000 times less well than CGRP does, and appears to be a weak partial agonist of AMY₁ rather than of CGRP receptors

Cys(Acm)2,7-h-alpha-CGRP differs from CGRP in its N terminal part, in particular the 7 first amino-acid of α-CGRP, is modified. In particular, the hydrogen atom of the sulfliydryl groups of the cysteine residues is substituted by an acetamidomethyl moiety. Cys(Acm)2,7-h-CGRP is therefore conformationally different from the native CGRP. Without being bound by theory, this difference seems to be responsible for the lack of activity of the compound toward CGRP receptor.

Furthermore, replacing the acetaminomethyl moiety (Acm) of Cys(Acm)2,7-h-alpha-CGRP by an ethylamide group (Et) yields the linear CGRP analogue Cys(Et)2,7-h-alpha-CGRP. Cys(Et)2,7-h-alpha-CGRP has a 10-fold reduction in affinity to the CGRP receptor, compared to CGRP, and is a partial agonist of both AMY₁ and CGRP receptors.

The difference in activity between Cys(Acm)2,7-h-CGRP and Cys(Et)2,7-h-alpha-CGRP are likely to be due to the difference in steric effects between the acetaminomethyl moiety and the ethylamide group. Thus, the N terminal part of CGRP, in particular the 7 first amino-acids of α-CGRP, appears to be of importance for activating the CGRP receptor. The Inventors have thus reasoned that compounds that comprise the 7 first amino-acid of α-CGRP will prove efficient agonists of CGRP receptors.

According to a particular embodiment, the agonist of the invention is a polypeptide that comprises or consists in the polypeptide of sequence SEQ ID No. 3: Ala-Cys-Asp-Thr-Ala-Thr-Cys, its functional variants and fragments.

CGRP and agonists of the CGRP receptor of the invention, their functional variants and fragments, can be synthesized for example by any suitable method, in particular techniques for obtaining peptides that are known in the art. Methods for obtaining peptides are indeed well known from the skilled person, and comprise chemical synthesis of peptides or recombinant DNA techniques.

CGRP and/or agonists of the CGRP receptor can be synthesized by a chemical synthesis method such as an Fmoc method (fluorenylmethyloxycarbonyl method) or a tBoc method (t-butyloxycarbonyl method), for example. In addition, the peptide of the present invention can also be synthesized using various types of commercially available peptide synthesizers. Other known methods for chemical synthesis include solid-phase techniques, partial solid-phase techniques, fragment condensation or classical solution addition. For example, the techniques of exclusively solid-phase synthesis are set forth in “Solid-Phase Peptide Synthesis”, Stewart et al., San Francisco, 1969, and are exemplified in U.S. Pat. No. 4,105,603. The fragment condensation method of synthesis is exemplified in U.S. Pat. No. 3,972,859.

Synthesis by recombinant DNA techniques, are well known from the person skilled in the art, and are further thoroughly detailed in Sambrook et al. (CSHL Press, 2001) and Ausubel et al. (John Wiley & Sons, 1988). The synthetic CGRP peptide may be obtained by transforming a microorganism using an expression vector including a promoter and operator together with said nucleotide sequence and causing such transformed microorganism to express the peptide. A non-human animal may also be used to produce the peptide using said nucleotide sequence and the general techniques set forth in U.S. Pat. No. 4,276,282.

When CGRP and/or agonists of the CGRP receptor are produced by recombinant DNA techniques, they are produced in the form of recombinant peptides. Generally, for producing a recombinant peptide, a nucleic acid polymer having a nucleotide sequence encoding the reference peptide is obtained, and it is then introduced into a preferred expression vector. Preferably, the nucleic acid polymer is a cDNA having a nucleotide sequence complementary to that of the nucleotide sequence of the mRNA coding for the reference peptide. The recombinant vector is then introduced in a host cell so as to produce said recombinant peptide.

A nucleic acid polymer having a nucleotide sequence encoding the reference peptide is preferably a cDNA having a nucleotide sequence complementary to that of the nucleotide sequence of the mRNA coding for the reference peptide. Said nucleotide sequence encoding the reference peptide may further comprise one or more sequences encoding a tag allowing purification of a protein, such as histidine (His) tag, or glutathione S-transferase.

By “vector”, it is herein referred to a plasmid or a virus useful for performing procedures of molecular biology and genetic recombination. A vector may have the following features: an origin of replication, a selectable marker gene, and a cloning site for the insertion of a gene. A vector may be engineered to contain regulatory sequences that act as enhancer and promoter regions and lead to efficient transcription of the gene carried on the expression vector. A nucleic acid of interest can be inserted into a vector able to replicate, that is to say a vector comprising an origin of replication, in order to amplify said nucleic acid, or to express the protein encoded by said nucleic acid. These vectors are better known as “cloning vectors” (to amplify a nucleic acid) or “expression vectors” (to express a protein), and are publicly available. Such vectors include, without limitation, plasmid vectors, cosmids, YACS, viral vectors (adenovirus, retrovirus, EBV episome), and phage vectors. According to the invention, an expression vector is a vector able to autonomously replicate in a host cell or able to be incorporated into the chromosome of a host cell and that is designed for protein expression in cells. An expression vector may comprises an origin of replication, a selectable marker, and a cloning site for the insertion of a gene, a promoter, a translation initiation sequence such as a ribosomal binding site and start codon, a termination codon, and a transcription termination sequence. Methods for inserting a nucleic acid polymer in vectors are known to those skilled in the art. Generally, a nucleic acid polymer is inserted into one or more restriction endonuclease site(s) using appropriate techniques known in the art, e.g. via ligation. It is additionally known to those skilled in the art that, depending on the nucleotide sequences present in the vector, said vector can replicate in different host cells, and/or the protein encoded by said nucleic acid can be expressed in different host cells.

By “host cell” it is herein referred to a prokaryotic or a eukaryotic cell in which the recombinant vector of the invention can be introduced, such as to amplify the nucleic acid as described above, and/or to express the protein encoded by said nucleic acid, for example any one of a bacterium, a yeast, an animal cell, or an insect cell. Examples of prokaryotic cells include, without limitation, bacteria such as Gram-negative bacteria of the genus Escherichia (e.g. E. coli RR1, LE392, X1776, W3110, DH5 alpha, JM109, KC8, K12), Serratia, Pseudomonas, Erwinia, Methylobacterium, Rhodobacter, Salmonella and Zymomonas, and Gram positive bacteria of the genus Corynebacterium, Brevibacterium, Bacillus, Arthrobacter, and Streptomyces.

Examples of eukaryotic cells include, without limitation, cells isolated from fungi, plants, and animals. Such cells notably include, yeasts of the genus Saccharomyces, cells of the fungi Aspergillus, Neurospora, Fusarium and Trichoderma, animal cells such as HEK293 cells, NIH3T3, Jurkat, MEF, Vero, HeLa, CHO, W138, BHK, COS-7, MDCK, C127, Saos, PC12, HKG, and insect cells Sf9, Sf21, Hi Five™ or of Bombyx mori. The use of insect cells is particularly described in the manual “Baculovirus Expression Vectors: A Laboratory Manual”, David R. O'Reilly et al. Oxford University Press, USA (1993). The expression vector may be introduced into a host cell according to any known method, depending on the type of such a host. Examples of such methods include, without limitation, electroporation, lipofection, calcium phosphate transfection, transfection using DEAE dextran, microinjection, and biolistics.

The host cell comprising the expression vector is cultured, and the recombinant peptide is then expressed and accumulates in the culture medium. After completion of the culture, the recombinant peptide may be isolated and purified from the culture of the transformant according to any common method of isolating and purifying a peptide known in the art.

Additionally, in order to facilitate or increase their delivery, CGRP or the agonist of the CGRP receptor can further be linked to a compound that target Langerhans cells.

In an embodiment of the invention, CGRP or the agonist of the CGRP is linked to a compound that targets Langerhans cells.

By “compound that targets Langerhans cells” it is herein referred to a compound that binds to molecules expressed on the plasma membrane of Langerhans cells. The skilled person may easily verify that a specific compound targets Langerhans cells by linking said compound to a fluorophore, incubate said fluorophore-linked compound with Langerhans cells and verify whether the fluorophore-linked compound targets Langerhans cells with any suitable technique known from the person of skills, such as fluorescence microscopy. Preferably, compounds that target Langerhans cells comprise for examples antibodies, peptides or sugars. More preferably, compounds that target Langerhans cells comprise for examples antibodies, peptides or sugars that bind to at least one molecule expressed on the plasma membrane of Langerhans cells.

An example of an antibody that targets Langerhans cells is the monoclonal antibody DCGM4 (commercially available from Beckman Coulter). Examples of a sugars that target Langerhans cells are Glcβ1-3Glcβ1-3Glc or Galα1-3(Fucα1-2)Gal, described in Feinberg, (J. Mol. Biol. 405, 1027-1039; 2011), compounds containing terminal mannose, terminal N-acetylglucosamine and 6-sulfogalactose residues, or blood-type antigens H, A and B (as described in Holla et al. (Protein Eng Des Sel., 24(9):659-69, 2011).

In an embodiment of the invention, CGRP or the agonist of the CGRP is linked to the monoclonal antibody DCGM4.

In another embodiment of the invention, CGRP or the agonist of the CGRP is linked to the compound Glcβ1-3Glcβ1-3Glc or to the compound Galα1-3(Fucα1-2)Gal.

Methods for linking two molecules are well known in the art and do not need a thorough explanation herein. For example, CGRP or the agonist of the CGRP receptor can be linked to a compound that targets Langerhans cells according to any known technique, such as for example by covalent binding between CGRP or the agonist of the CGRP receptor and the compound that targets Langerhans cells. According to the invention, the terms “HIV infection” refers to a viral infection by at least one of the variants of the Human Immunodeficiency Virus (HIV). By viral infection it is herein referred to the invasion of a human subject's bodily tissues by a virus, its multiplication, and the reaction of the human subject's tissues to this virus. HIV infection can easily be measured from a biological sample of a human subject by any appropriate ELISA technique and/or kit. Advantageously, the kits are adapted to detect antibodies to HIV-1. Even more advantageously, the kits are adapted to detect the antigen p24 of HIV (hereafter “p24”) (ref Genebank: AAB82827.1). The protein p24 is a core protein in the HIV virus particle. It forms the “capsid” of the virus, the case in which HIV's RNA genome is kept. It has been shown that the level of p24 found in the blood serum is an indicator of HIV progression. More precisely, an increase in the p24 content found in blood serum correlates with an increase of the HIV load in a subject. A virus load is a measure of the severity of a viral infection, and can be calculated by estimating the amount of virus in an involved body fluid. Preferably, the variant of HIV is chosen in the HIV-1 group or HIV-2 group of HIV variants. As mentioned in the introduction, HIV-1 is the cause of the majority of HIV infections globally. Yet preferably, the variant of HIV is chosen in HIV-1 group of HIV variants. Thus, the prevention of an HIV infection by CGRP or an agonist of the CGRP receptor can easily be verified by such ELISA technique and/or kit. According to the invention, “CGRP and the agonist of CGRP receptor prevent an HIV infection in a subject” means that an HIV infection cannot be detected in said subject treated with CGRP and the agonist of CGRP receptor.

The Inventors have further deciphered the biological pathway associated with CGRP activity in preventing HIV infection. It was already known that HIV gains access into the body mainly during sexual intercourse, by crossing epithelial barriers that cover mucosal surfaces of the genital tracts. In these epithelia, the first cells targeted by HIV-1 are Langerhans cells that are located in close proximity to the mucosal surface. Langerhans cells bind and internalize HIV, followed by local transmission of the internalized intact virions to TCs. Such viral transfer induces extensive replication of the virus in TCs and results in local expansion of a local founder population of HIV infected cells, which is a prerequisite for HIV dissemination and systemic infection. The Inventors have shown that CGRP i) induces HIV degradation within Langerhans cells, ii) diminishes the LC-TC conjugate formation iii) diminishes the adhesive potential of Langerhans cells, in particular to TCs by decreasing the surface expression of several integrins (such as CD29, CD49e and CD50), iv) increases cell surface expression of langerin, v) activates NFκB which results in an increase of the secretion of the anti-HIV chemokine-CCL3/MIP-1α by Langerhans cells. All of those different parameters, either taken separately or together, are hallmarks of the activity of a CGRP receptor ligand in preventing HIV transfer from Langerhans cells to TCs both in vitro and in vivo. It will be clear for the skilled person that a compound able to prevent the in vitro HIV transfer from Langerhans cells to TCs is capable of the same in vivo.

All of those markers can therefore be used as markers of the activity of a compound in the prevention of HIV infection in humans. Active compounds that present the same characteristics as CGRP, and therefore that are useful for the prevention of an HIV infection in a human subject, can be selected based on those parameters.

A second object of the invention is a method for selecting an active compound for the prevention of an HIV infection in a human subject, comprising the steps of:
a) contacting Langerhans cells with a candidate compound;
b) in vitro infecting of Langerhans cells by at least one HIV variant,
c) measuring of at least one of the following parameters:
   HIV content,
   adhesive potential of Langerhans cells, in particular to TCs,
   secretion of anti-HIV chemokines by Langerhans cells.

According to the invention, the terms “Langerhans cells” (also called “LCs”) refers to dendritic cells of the skin and mucosa that contain large granules called Birbeck granules. Birbeck granules are rod or “tennis-racket” shaped cytoplasmic organelles with a central linear density and a striated appearance, which presence in a cell can easily be observed with electronic microscopy. Langerhans cells are present in all layers of the epidermis, as well as in the mucosa of the mouth, foreskin, glans, fossa navicularis and vagina and exocervix. According to the invention, Langerhans cells have functional CGRP receptor on their surface.

In the context of the invention “Langerhans cells” refers to both primary Langerhans cells and immortalized Langerhans cells, such as for example Langerhans cells from cell lines. Such cell lines are well known from the skilled person and comprise for example the human CD34+ acute myeloid leukemia cell line MUTZ-3, which can be differentiated into Langerhans cells following treatment with specific cytokines such as described in de Jong et al. (J leuk Biol, 87(4):637-43, 2010)

By immortalized cells it is herein referred to cells dividing beyond the Hayflick limit. The Hayflick limit (or Hayflick phenomenon) is the number of times a normal human cell population will divide until cell division stops. Cells can be immortalized due to specific diseases, such as cancer cells, or as a result of the implementation of a immortalization technique. Immortalization techniques are well known to those skilled in the art that can easily choose among them the one best suited for its purpose. For example, and without limiting the invention to these examples, one can cite as immortalization technique: transformation by an oncogene, such as the SV40 T antigen, the ras protein, the myc protein, the abl protein, or overexpression of a telomerase reverse transcriptase, for example hTERT, or culture of cells at confluence for several passages. All those techniques are classic cell biology techniques which do not need to be further detailed herein. Preferably, Langerhans cells are from human origin. For example, Langerhans cells are originated from biological samples from human subject, and preferentially immortalized by an immortalization technique.

According to the invention, the term “biological sample” refers to a subset of biological tissues from an organism, its cells or component parts (e.g., body fluids, including but not limited to, blood, mucus, lymphatic fluid, synovial fluid, cerebrospinal fluid, saliva, amniotic fluid, amniotic cord blood, urine, vaginal fluid and semen). “A biological sample” further refers to a homogenate, lysate or extract prepared from a subset of biological tissues from an organism, its cells or component parts, or a fraction or portion thereof, including but not limited to, for example, blood, blood cells, plasma, serum, spinal fluid, lymph fluid, the external sections of the skin, respiratory, intestinal, and genitourinary tracts, tears, saliva, milk, organs. Most often, the sample has been removed from a subject.

According to the invention, the term “infecting in vitro” refers to an in vitro technique wherein in vitro cultured cells are put in contact with an amount of viral strain sufficient for infecting said cells. The amount of viral strain sufficient for infecting said cells is also commonly said to enable efficient viral transfer. The amount of viral strain sufficient for infecting cells can easily be estimated by the skilled person by using the median tissue culture infective dose (TCID₅₀). TCID₅₀ is the amount of a pathogenic agent that produces pathological change in 50% of cell cultures inoculated, and is expressed as TCID₅₀ per volume units of cell culture (usually ml). The TCID₅₀/ml of a specific viral strain can be calculated by the following method: host cells are plated and serial dilutions of the virus are added; after incubation, the percentage of cell death (i.e. infected cells) is observed and recorded for each virus dilution, and results are used to calculate the TCID₅₀ result per ml of cell culture.

According to the invention, the term “HIV variant” refers to any a viral strain that can be classified into any of the HIV groups, subgroups, types, and/or subtypes. Preferably, an HIV variant according to the invention belongs to the HIV1 or the HIV2 group. Preferably, the HIV variant according to the invention belongs to the HIV1 group. More preferably, the HIV variant may be classified into the R5 tropism. Even more preferably, the HIV variant is JRCSF or ADA of Glade B and R5 tropism.

According to the invention, the HIV content may be measured by any method known to the skilled person. For example, HIV content can be measured indirectly by measuring p24 levels. The protein p24 is a core protein in the HIV virus particle and therefore the level of p24 directly correlates with the amount of VIH viruses.

Moreover, the Inventors have shown that the level of langerin present on the surface of Langerhans cells inversely correlates with the amount of HIV particles associated with Langerhans cells. Langerin (NCBI ref.: NP_056532.3) is a protein encoded by the CD207 gene in humans. Langerin is a type II transmembrane cell surface receptor expressed by Langerhans cells. At a dose of HIV-1 of HIV-1 TCID 100 (MOI 5×10⁻³, 1.1 ng p24), langerin was shown to bind to HIV-1 and subsequently internalize it into Birbeck granules to be degraded. Thus, it is possible to measure the HIV degradation by measuring the p24 level and/or by measuring the langerin expression level on the surface of Langerhans cells membrane. Thus, in the context of the invention, determining the HIV degradation is preferably achieved by measuring p24 level and/or by measuring the langerin expression level on the surface of Langerhans cells, which inversely correlate

According to the invention, the term “HIV content” refers to the content in HIV viruses in cell lysates of Langerhans cells after in vitro HIV infection

The HIV content can easily be measured by measuring p24 level. According to the invention, the terms “p24 level” and “p24 concentration” are equivalent. The p24 level can easily be detected by the skilled person by any immunological technique known from the skilled person. In order to measure the p24 level, the skilled person may use any kit available, such as for example the Innotest HIV antigen mAb Elisa kits (Innogenetics). Preferably, the skilled person will refer to the method described in Witte et al. (Nat Med 13, 367-71 (2007)), wherein the HIV content is measured by p24 ELISA.

The HIV content is inversely correlated with langerin expression level. According to the invention, the terms “langerin expression level” refer to the quantity of langerin protein (NCBI ref.: NP_056532.3), which is expressed on the surface of Langerhans cells. The langerin expression level can easily be detected by the skilled person by any immunological technique known from the skilled person, particularly flow cytometry analysis following immunostaining, that is to say staining of an antigen via the use of an antibody specific for said antigen.

By “antibody specific for” is herein referred to any molecule, preferably whole immunoglobulin molecule that binds selectively and reversibly to an antigen with the required selectivity. The term refers to both intact immunoglobulin molecules, such as monoclonal and polyclonal antibodies, as well as bi-specific antibodies, humanized antibodies, chimeric antibodies, anti-idiotypic (anti-ID) antibodies, single chain, Fab fragments, F (ab′), the fusion proteins, as they bind selectively and reversibly to said antigen.

By “selectively binds” it is herein referred to the ability of antibodies to preferentially bind to an antigen, in comparison with other antigens. The interaction of antibodies with an antigen can be characterized in terms of a binding affinity, which is commonly expressed by the affinity constant. The affinity constant (also known as the association constant), Ka, is a numerical constant used to describe the binding affinity of two molecules at equilibrium. Preferably, an antibody will be said specific for an antigen when the binding affinity of said antibody for said antigen will be superior than the binding affinity of the same antibody for unrelated antigens.

The binding affinity can be measured using a variety of methods known to those skilled in the art including immunoblotting, immunoprecipitation analyzes, Radio-Immuno Assays, ELISAs, assays of antibodies by immunofluorescence microscopy, surface plasmon resonance (BiaCORE). Preferably, the affinity of an antibody specific for an antigen has an affinity constant of between about 10³ M⁻¹ and about 10¹² M⁻¹ for said antigen.

Techniques to produce polyclonal or monoclonal antibodies specific for a polypeptide and/or for said peptide fragments are well known to those skilled in the art and need not be described in detail herein. Polyclonal antibodies can be obtained by immunization, possibly by multiple immunizations, of an animal with said polypeptide, followed by recovery of serum from said animal and purification of the desired antibodies, in particular by affinity chromatography using the polypeptide used for the immunization. Monoclonal antibodies may be obtained by the hybridoma method described in Köhler et al. (Nature, 1975, 256 (5517): 495-497).

Methods for preparation and use of antibodies, and the assays mentioned herein before are described in Harlow, E. and Lane, D. Antibodies: A Laboratory Manual, (1988), Cold Spring Harbor Laboratory Press.

According to the invention, immunostaining comprises direct immunostaining and indirect immunostaining. In direct immunostaining, an antibody directly labeled using an enzyme or a fluorophore is used to detect a specific protein epitope. These antibodies can be monoclonal or polyclonal. Direct immunostaining preferably uses monoclonal antibodies. In indirect immunostaining, a primary antibody, specific for the antigen, is recognized by a high affinity binding partner, which can be a secondary antibody, which is linked to an enzyme or fluorophore. For example, the biotin-streptavidin is one commonly used high affinity interaction. The primary antibody can be probed for using a broader species-specific secondary antibody that is labeled using an enzyme, or fluorophore. Preferably, detecting the langerin expression level is performed with the use of an antibody specific for langerin, particularly human langerin. Antibodies specific for langerin are available commercially, such as for example the monoclonal antibody DCGM4 (Beckman Coulter). Preferably, the skilled person will use antibodies specific for langerin that are able to recognize the native langerin on the cell surface of Langerhans cells. In order to verify that a given antibody, specific of langerin, is able to recognize the native langerin on the cell surface of Langerhans cells, the skilled person may for example check by flow cytometry analysis that the given antibody can recognize said langerin either on non lysed Langerhans cells or other cell lines expressing langerin. At this end, the skilled person may for example use any cell line transfected with a vector expressing the protein langerin, preferably tagged with a fluorophore.

According to the invention, the adhesive potential of Langerhans cells, in particular to TCs, may be measured by any method known to the skilled person. For example, the skilled person may measure the amount of proteins known to be involved in cell adhesion, in particular cell-cell adhesion, such as proteins of the integrin family and proteins of the intercellular adhesion molecule (ICAM) family, on the membrane of Langerhans cells. The Inventors have found that the adhesive potential of Langerhans cells can be determined by measuring the level of the membrane proteins CD29, CD49e and CD50 expressed on the surface of Langerhans cells. CD29 and CD49e are proteins of the integrin family involved in cell adhesion. CD50 is a protein of the ICAM family involved in cell adhesion. More particularly, the level of CD50 expressed on the surface of Langerhans cells is directly related to the adhesive potential of Langerhans cells to TCs. Thus, in the context of the invention, determining the adhesive potential of Langerhans cells is preferably achieved by measuring CD29, CD49e and/or CD50 expression level on the surface of Langerhans cells membrane. More preferably, determining the adhesive potential of Langerhans cells is achieved by measuring CD50 expression level on the surface of Langerhans cells membrane.

According to the invention, the term “CD29 expression level” refers to the quantity of CD29 protein (NCBI ref.: NP_002202.2), which is expressed on the surface of Langerhans cells.

According to the invention, the term “CD49e expression level” refers to the quantity of CD49e protein (NCBI ref.: NP_002196.2), which is expressed on the surface of Langerhans cells.

According to the invention, the term “CD50 expression level” refers to the quantity of CD50 protein (NCBI ref.: NP_002153.2), which is expressed on the surface of Langerhans cells.

The CD29, CD49e and/or CD50 expression level can easily be detected by the skilled person by any immunological technique known from the skilled person, particularly ELISAs and flow cytometry analysis following immunostaining, such as described above.

Preferably, detecting the CD29 expression level is performed with the use of an antibody specific for CD29, particularly human CD29. Antibodies specific for CD29 are available commercially, such as for example the monoclonal antibody MEM-101A (ImmunoTools). Preferably, detecting the CD49e expression level is performed with the use of an antibody specific for CD49e, particularly human CD49e. Antibodies specific for CD49e are available commercially, such as for example the monoclonal antibody NKI-SAM-1 (ImmunoTools). Preferably, detecting the CD50 expression level is performed with the use of an antibody specific for CD50, particularly human CD50. Antibodies specific for CD50 are available commercially, such as for example the monoclonal antibody MEM-171 (ImmunoTools).

The adhesive potential of Langerhans cells to TCs can also be measured by measuring conjugate formation between Langerhans cells and TCs. The measure of conjugate formation between Langerhans cells and TCs can be realized according to any technique known from the skilled person. In particular, the measure of conjugate formation between Langerhans cells and TCs can be realized according to the method disclosed herein in the experimental part.

According to the invention, measuring the secretion of anti-HIV chemokines by Langerhans cells, may encompass measuring the secretion of any anti-HIV chemokines known to the skilled person. In the context of the invention, the term anti-HIV chemokine means any chemokine known to impede infection of CD4+ TCs by at least one HIV variant, such as, for example, the CCR5-binding chemokines CCL3/MIP-1α, CCL4/MIP-1β and CCL5/RANTES, all of which block HIV-1 infection by binding the HIV-1 co-receptor CCR5 on CD4+ TCs. In a preferred embodiment, measuring the secretion of anti-HIV chemokines by Langerhans cells comprises measuring the expression level of at least one of CCL3/MIP-1α, CCL4/MIP-1β and CCL5/RANTES. More preferably, measuring the secretion of anti-HIV chemokines by Langerhans cells comprises measuring the expression level of CCL3/MIP-1α. According to the invention, the secretion of anti-HIV chemokines by Langerhans cells, may be measured by any method known to the skilled person.

According to the invention, the term “CCL3/MIP-1α level” refers to the quantity of CCL3/MIP-1α (NCBI ref: NP_002974.1), in the co-culture medium. According to the invention, the term “CCL4/MIP-1β level” refers to the quantity of CCL4/MIP-1β (NCBI ref.: NP_002975.1) in the co-culture medium. According to the invention, the term “CCL5/RANTES” refers to the quantity of CCL5/RANTES (NCBI ref: NP_002976.2) in the co-culture medium. The CCL3/MIP-1α, CCL4/MIP-1β and/or CCL5/RANTES level can easily be measured by the skilled person by any immunological technique known from the skilled person, particularly ELISAs and flow cytometry, from a sample of said co-culture medium.

Methods and kits for measuring the levels of anti-HIV chemokines are well known to the skilled person, and commercially available, such as for example the FlowCytomics multiplex kit (Bender MedSystems)

To increase the chances to obtain particularly efficient compounds for the prevention of an HIV infection, the skilled person can further screen candidate compounds for their actual efficacy in reducing the HIV replication in TCs. Toward this end, the Inventors have set up an embodiment of the method for selecting an active compound for the prevention of an HIV infection in a human subject of the invention, based on the measurement of those parameters in an in vitro model of a co-culture of HIV infected Langerhans cells and TCs.

In another embodiment, the method of the invention for selecting an active compound for the prevention of an HIV infection in a human subject comprises the steps of:
a) contacting Langerhans cells with a candidate compound;
b) in vitro infecting of Langerhans cells from step a) by at least one HIV variant,
c) growing infected Langerhans cells from step b) with CD4+ T cells in culture medium,
d) measuring at least one of the following parameters:
   HIV content
   adhesive potential of LCs, in particular to TCs, or
   secretion of anti-HIV chemokines by LCs.

According to the invention, the term “T cells” (TCs) refers to a subgroup of lymphocytes cells. T cells can be distinguished from other lymphocytes, such as B cells and natural killer cells (NK cells), by the presence of a T cell receptor (TCR) on their cell surface. There are several subsets of T cells, each with a distinct function (see for example Goldsby R A, Kindt T K, Osborne B A and Kuby J (2003) Immunology, 5th Edition, W.H. Freeman and Company, or Janeway C A, Travers P, Walport M, and Shlomchik M (2001) Immunobiology, 6th Edition, Garland Publishing).

According to the invention, the term “CD4+ T cells” refers to a subset of lymphocyte T cells that express the surface protein CD4. According to the invention, CD4 is a glycoprotein found on the surface of immune cells, in particular T cells. In humans, the CD4 protein (NCBI reference: NP_000607.1) is encoded by the CD4 gene (NCBI reference: NG_027688.1). CD4+ T cells can easily be isolated from a biological sample, preferably blood, with any technique or kit available, such as the Human CD4+ T cell enrichment kit (StemCell).

In order to facilitate selection of active compounds, the skilled person can advantageously compare the measurements of the parameters of the method with reference values.

In a particular embodiment, the method of the invention for selecting an active compound for the prevention of an HIV infection in a human subject is characterized in that it further comprises at least one of the following steps:
i. The HIV content is compared to a HIV content threshold;
ii. The adhesive potential of Langerhans cells is compared to an adhesive potential of Langerhans cells threshold;
iii. The secretion of anti-HIV chemokines by Langerhans cells is compared to a secretion of anti-HIV chemokines threshold;

In the context of the invention, a threshold is a reference value to which the measured parameter is compared in order to determine if the candidate compound is an active compound. In particular, a threshold is a value obtained when a reference compound is used as the candidate compound of step a) of the method for selecting active compound of the invention, or a normalized value calculated from one or more values obtained when a reference compound is used as the candidate compound of step a) of the method for selecting active compound of the invention. Preferably, the threshold of a given parameter is calculated based on more than one measurement of said parameter obtained when a reference compound is used as the candidate compound of step a) of the method for selecting active compound of the invention. More preferably, the threshold of a given parameter is calculated based on 2, 3, 4, 5, 10 measurements of said parameter obtained when a reference compound is used as the candidate compound of step a) of the method for selecting active compound of the invention.

According to the invention, the term “HIV content threshold” refers to the average HIV content obtained when a reference compound is used as the candidate compound of step a) of the method for selecting active compound of the invention.

According to the invention, the term “adhesive potential of Langerhans cells threshold” refers to the average adhesive potential of Langerhans cells obtained when a reference compound is used as the candidate compound of step a) of the method for selecting active compound of the invention.

According to the invention, the term “secretion of anti-HIV chemokines threshold” refers to the average secretion of anti-HIV chemokines by Langerhans cells obtained when a reference compound is used as the candidate compound of step a) of the method for selecting active compound of the invention.

Preferably, the reference compound is a compound that exhibit interesting properties in the prevention of an HIV infection in a human subject. More preferably, the reference compound is CGRP.

When CGRP is used in the method of the invention as a reference compound, the candidate compound is selected as an active compound for the prevention of an HIV infection in a human subject if:
i. The HIV content is equal or inferior to a HIV content threshold; or
ii. The adhesive potential of Langerhans cells is equal or inferior to an adhesive potential of Langerhans cells threshold; or
iii. The secretion of anti-HIV chemokines by Langerhans cells is equal or superior to a secretion of anti-HIV chemokines threshold.

The skilled person can thus decide to take into consideration one or several parameters.

In order to increase the chance to select the most efficient and appropriate active compounds, the skilled person may decide to take all of the 4 parameters i to iv into consideration.

In a particular embodiment, the method of the invention for selecting an active compound is characterized in that said candidate compound is selected as an active compound for the prevention of an HIV infection in a human subject if:
i. The HIV content is compared to a HIV content threshold; and
ii. The adhesive potential of Langerhans cells is compared to an adhesive potential of Langerhans cells threshold; and
iii. The secretion of anti-HIV chemokines by Langerhans cells is compared to a secretion of anti-HIV chemokines threshold.

In that particular embodiment, when CGRP is used in the method of the invention as a reference compound, the candidate compound is selected as an active compound for the prevention of an HIV infection in a human subject if:
i. The HIV content is equal or inferior to a HIV content threshold; and
ii. The adhesive potential of Langerhans cells is equal or inferior to an adhesive potential of Langerhans cells threshold; and
iii. The secretion of anti-HIV chemokines by Langerhans cells is equal or superior to a secretion of anti-HIV chemokines threshold.

In an embodiment, the candidate compound is an agonist of the CGRP receptor.

According to another aspect of the invention, CGRP or agonist of the CGRP receptor is administered through any route of administration, such as for example oral, parenteral, topical administration.

Because the compounds of the invention target specifically the interaction between Langerhans cells and TCs, and thus the very first steps of HIV infection, they can be administered directly locally, onto the skin or various mucosa, including the male and female genital tracts, and thus do not require systemic administration.

Therefore, the CGRP or agonist of the CGRP receptor is preferably administered topically. By topical administration, it is herein referred to mucosal or cutaneous administration. By mucosal it is herein referred to vaginal, buccal, rectal, intestinal (i.e., mucosa of the small and large intestines), tracheal, bronchial, pharyngeal, gastric, nasal and penile mucosa. Preferably, the administration through mucosal route comprises administration through oral mucosa, vaginal mucosa, penile mucosa and/or rectal mucosa.

In order to facilitate their administration, it is advantageous to formulate the compounds of the invention into compositions.

Another object of the invention is therefore a composition comprising CGRP or agonist of the CGRP receptor for use in the prevention of an HIV infection in a human subject. The composition of the invention is advantageously formulated specifically for the intended administration route. Thus, according to the invention, the composition comprises CGRP or agonist of the CGRP receptor and a pharmaceutically acceptable carrier. According to the invention, “pharmaceutically acceptable carrier” means a compound, or a combination of compounds, contained in a pharmaceutical composition, that does not cause secondary reactions and that, for example, facilitates administration of the active compounds, increases its lifespan and/or effectiveness in the organism, increases its solubility in solution or improves its storage. Such pharmaceutical carriers are well-known and will be adapted by a person skilled in the art according to the nature and the administration route of the active compounds selected.

In an embodiment, the composition of the invention is formulated for cutaneous administration.

Cutaneous administration can be obtained by formulating the CGRP or agonist of the CGRP receptor into solid, liquid or semi-solid forms. Among solid forms for cutaneous administration, one can cite for example powders, aerosols, plasters. Among liquid forms for cutaneous administration, one can cite for example lotions, liniments, solutions, emulsions, suspensions. Among semi-solid forms for cutaneous administration, one can cite for example ointments, creams, paste, gels, jelly, and suppositories. When formulated for cutaneous administration, particularly when formulated into liquid or semisolid forms, the composition of the invention comprises a carrier selected from the group consisting in solutions, emulsions, microemulsions, oil-in-water emulsions, anhydrous lipids and oil-in-water emulsions, other types of emulsions.

It is well known to the skilled person that absorption of an active compound by the skin may be enhanced either by chemical enhancers such as solvents or surfactants. Those compounds are on the other hand not recommended for mucosal administration, because of their potentially irritant properties.

In a particular embodiment, the composition of the invention formulated for cutaneous administration further comprises at least one solvent and/or at least one surfactant.

Solvents may be chosen from the list consisting of water, alcohols, methanol and ethanol, alkyl methyl sulfoxide, dimethyl sulfoxide, alkyl homologs of methyl sulfoxide, dimethyl acetamide and dimethylformamide, pyrrolidones-2-pyrrolidone, N-methyl, 2-pyrrolidone, laurocapram (Azone), miseellancous solvents-propnylene glycol, glycerol, silicone fluids, isopropyl palmitate.

Surfactant may be chosen from the list consisting of Dioctyl sulphosuccinate, Sodium lauryl sulphate, Decodecylmethyl sulphoxide, Pluronic F127, Pluronic F68, urea, N,N-dimethyl-m-toluamide, calcium thioglycolate.

In another embodiment, the composition of the invention is formulated for administration through oral mucosa, and/or for administration through vaginal and/or penile and/or rectal mucosa.

Those of ordinary skill in the clinical arts will be familiar with formulations and vehicles for drug delivery into mucosa. Useful references in this regard are Chien (Novel Drug delivery system, Chapters 3 through 6 and 9, Marcel Dekker, 1992), and Pharmaceutical Dosage Forms and Drug Delivery Systems (ANSEL et al., 1994, WILLIAMS & WILKINS).

Administration through oral mucosa can be obtained by formulating the CGRP or agonist of the CGRP receptor into inhalables, sprays and the like (e.g., aerosol spray or pump spray and the like), solutions, or as gels. When formulated for administration through oral mucosa, the composition of the invention comprises a vehicle selected in the group comprising solutions, emulsions, microemulsions, oil-in-water emulsions, anhydrous lipids and oil-in-water emulsions, other types of emulsions.

Administration through vaginal and/or penile and/or rectal mucosa can be obtained by formulating the CGRP or agonist of the CGRP receptor into solution, enema, foam, suppository, vaginal tablet or topical gel. When formulated for administration through vaginal and/or penile and/or rectal mucosa, the composition of the invention comprises a vehicle selected in the group comprising hydrophilic and hydrophobic vehicles such as those commonly used in formulating emulsion or gel preparations (e.g., oil/water emulsion gel).

Preferably, the composition of the invention comprises an effective amount of CGRP and/or agonist of the CGRP receptor. According to the present invention, an “effective amount” of CGRP and/or agonist of the CGRP receptor is one which is sufficient to achieve the desired biological effect, in this case the prevention of an HIV infection. It is understood that the effective amount will be adapted by the skilled person according to the usual criteria such as for example the age, sex, health of the subject, and surface of skin or mucosa on which the compounds are to be administered.

CGRP and/or an agonist of the CGRP receptor may be used in a preventive treatment that comprises a onetime administration, for example prior to intercourse, or consecutive administrations, for example in view of long-term preventive treatments.

CGRP provoked migraine attacks are due to its vasodilation properties, and are associated with dilation of both the middle meningeal artery (MMA), a major artery that supplies blood to a membrane (dura) that envelops the brain, and the middle cerebral artery (MCA). Several approaches are possible to diminish the potential side-effects of the compounds of the invention.

These side-effects can be diminished by following a specific treatment scheme, more precisely by making sure that the consecutive administrations are separated by enough time without CGRP and/or agonist of the CGRP receptor treatment. In a particular embodiment, the consecutive administrations of CGRP and/or agonist of the CGRP receptor are separated by at least 1 day, preferably 2 days, yet preferably 5 days.

The composition of the invention can also advantageously be formulated in order to release CGRP and/or agonist of the CGRP receptor in the subject in a timely controlled fashion. In a particular embodiment, the composition of the invention is formulated for controlled release of CGRP and/or agonist of the CGRP receptor. Controlled release can be achieved by the use of specific formulations such as patches or adhesive compositions. Specific formulations for the controlled release of a drug or medicament from patches, bandages, dressings and different kinds of adhesive compositions applied to the skin are described in U.S. Pat. No. 3,577,516, U.S. Pat. No. 3,579,628, U.S. Pat. No. 3,734,097, U.S. Pat. No. 4,292,299, U.S. Pat. No. 4,059,686, U.S. Pat. No. 4,226,848, U.S. Pat. No. 4,374,126, U.S. Pat. No. 4,409,206, U.S. Pat. No. 4,490,322, and U.S. Pat. No. 4,310,509.

The side effects of CGRP and/or agonist of the CGRP receptor can also be diminished by the complementary use of a compound with vasoconstriction properties. In a particular embodiment, the composition of the invention further comprises at least a compound chosen from amphetamines, antihistamines, methylphenidate, mephedrone, oxymetazoline, phenylephrine, propylhexedrine, pseudoephedrine, caffeine, tetrahydrozoline hydrochloride, and/or psilocybin.

The Inventors have further discovered that CGRP can be used as a biomarker for the in vitro diagnosis of an HIV infection in a human subject. More particularly, the Inventors have shown that subjects infected with HIV have a level of CGRP that is lower than the CGRP level in non-infected subjects.

A fourth object of the invention is a method for the in vitro diagnosis of an HIV infection in a human subject, characterized in that it comprises determining the level of CGRP from a biological sample from said subject.

According to the invention, the term “determining the level of Calcitonin Gene-Related Peptide” means determining the level of at least one of the peptides α-CGRP, β-CGRP, and their functional variants and fragments, as defined above. Preferably, the term “determining the level of Calcitonin Gene-Related Peptide” means determining the level of at least one of the peptides α-CGRP and β-CGRP. Yet preferably, the term “determining the level of Calcitonin Gene-Related Peptide” means determining the level of the peptide α-CGRP.

Methods for determining the level of polypeptides in biological samples are well known in the art and include immunological techniques, among which Western-blotting, ELISAs and Radio-Immuno Assays, or various proteomics techniques such as mass spectrometry or chromatography.

Preferably, determining the level of CGRP is achieved by immunological techniques. Yet preferably, determining the level of CGRP is achieved by Western-blotting, ELISAs and/or Radio-Immuno Assays.

Immunological techniques are based on the use of molecules able to recognize an antigen with high specificity and high sensitivity. Most commonly antibodies specific for the antigen are used for this purpose.

A large number of antibodies specific for CGRP are currently available on the market. For example the skilled person may use any ELISA kit available, such as for example the human CGRP Peptide Enzyme Immunoassay (Peninsula).

Preferably, in the method for the in vitro diagnosis of an HIV infection of the invention the biological sample is chosen from blood, plasma and/or serum. Yet preferably, the biological sample is plasma.

Preferably, the method for the in vitro diagnosis of an HIV infection in a human subject of the invention comprises the following steps:
a) determining the level of CGRP from a biological sample from said subject; and
b) concluding to an HIV infection in said subject if the level of CGRP from step a) is inferior to the normal level of CGRP in the biological sample from non-infected human subjects.

According to the invention, the term “normal level of CGRP in the biological sample from non-infected human subjects” refers to the normalized level of CGRP in biological sample from non-infected human subjects. The normal level of CGRP can easily be calculated by the skilled person, based on the level of CGRP in biological samples from at least 1, 2, 5, 10, 20 non-infected subjects. According to the invention the terms “non-infected human subjects” refer to human subjects that have been diagnosed as not infected by neither HIV-1 nor HIV-2. For the purpose of diagnosing that a human subject is not infected by neither HIV-1 nor HIV-2, the skilled person may use any diagnosis method known in the art, such as for example the Innotest HIV antigen mAb Elisa kit (Innogenetics).

Moreover, the inventors have found that subjects infected with HIV and receiving Highly Active Antiretroviral Therapy (HAART) have a level of CGRP similar to the CGRP level in non-infected subjects.

A fifth object of the invention is an in vitro method for assaying the efficiency of Highly Active Antiretroviral Therapy in the treatment of HIV infection in a subject, characterized in that it comprises determining the level of CGRP from a biological sample from said subject.

Highly Active Antiretroviral Therapies are treatment regimens commonly administered in order to suppress HIV viral replication and the progression of HIV disease. The usual Highly Active Antiretroviral Therapy combines two or more different molecules such as two nucleoside reverse transcriptase inhibitors (NRTIs) and a protease inhibitor (PI), two NRTIs and a non-nucleoside reverse transcriptase inhibitor (NNRTI) or other such combinations. Highly Active Antiretroviral Therapies have proven to reduce the amount of active virus and in some cases can lower the number of active virus until it is undetectable by current blood testing techniques.

According to the invention, the terms “Highly Active Antiretroviral Therapy” refers to a combination of molecules used in the treatment or prevention of an HIV infection in a human subject. Highly Active Antiretroviral Therapies are well known from the skilled person. Preferably, the terms “Highly Active Antiretroviral Therapy” refers to at least one of the following combination of molecules:
zidovudine and lamivudine;
abacavir and zidovudine and lamivudine;
lopinavir and ritonavir;
abacavir and lamivudine;
efavirenz and tenofovir and emtricitabine;
rilpivirine and tenofovir and emtricitabine;
elvitegravir and cobicistat and tenofovir and emtricitabine.

Preferably, the in vitro method of the invention for assaying the efficiency of Highly Active Antiretroviral Therapy in the treatment of HIV infection in a subject, comprises the following steps:
a) determining a first level of CGRP from a first biological sample from said subject; and
b) determining a second level of CGRP from a second biological sample from said subject; and
c) concluding that the Highly Active Antiretroviral Therapy is efficient in the treatment of HIV infection in a subject if the level of CGRP from step a) is inferior to the level of CGRP from step b).

It will be clear to the skilled person that the first biological sample refers to a biological sample collected prior to the second biological sample. Preferably, the first biological sample is collected from the subject at least 10, 20, 30, 40, 50, 60, 70, 80, 90, 120, 150, 180 days prior to the second biological sample. In an embodiment, the first biological sample is collected before the first administration of Highly Active Antiretroviral Therapy to the subject, and the second biological sample is collected after the first administration of Highly Active Antiretroviral Therapy to the subject.

It is to be understood that “determining the level of Calcitonin Gene-Related Peptide” refers to the same definition as disclosed above, and can be achieved by the same above-mentioned techniques.

Preferably, in the method for the in vitro diagnosis of an HIV infection of the invention the biological sample is chosen from blood, plasma and/or serum. Yet preferably, the biological sample is plasma.

### EXPERIMENTAL RESULTS

### CGRP Inhibits HIV-1 Transfer from LCs to TCs

### Cells Preparation

Peripheral blood mononuclear cells (PBMCs) from healthy donors were separated from whole blood by a standard Ficoll gradient. CD14+ monocytes and CD4+ TCs were obtained from PBMCs by negative magnetic selection (Stemcell Technologies, Grenoble, France) according to the manufacturer's instructions. MDLC were prepared and maintained as described (Masterson et al., Blood 100:701-703; Geissmann et al., J Exp Med 187:961-966.)

### MDLC and TC Co-Culture

MDLC (1×105) were pretreated with CGRP alpha (Sigma, St. Louis, Mo., USA), CGRP8-37 (Sigma, St. Louis, Mo., USA) or Cys(Acm)2,7 (Bachem, Bubendorf, Switzerland), washed, and pulsed with HIV-1 JRCSF (NIH) for 2 h (10 ng corresponding to 1000 TCID50; prepared as described (Ganor et al., Mucosal Immunol 3:506-522; Zhou, et al.; PLoS Pathog 7:e1002100.). The cells were then washed again and incubated for a week with TCs (3×105; autologous for MDLC). HIV-1 replication was measured in the co-culture supernatants a week later by p24 ELISA (Innotest; Innogenetics, Gent, Belgium).

### Results

CGRP-pretreatment of MDLCs resulted in a dose- and time-dependent inhibition of HIV-1 transfer to TCs, reaching maximal inhibition of 73.0% following pretreatment with 100 nM CGRP for 24 h (FIG. 1). Similar inhibition was observed when MDLCs were pulsed with another Glade B/R5 HIV-1 molecular clone (ADA; data not shown). Similar CGRP-pretreatment of TCs did not affect HIV-1 transfer (FIG. 1). The inhibitory effect of CGRP was completely abrogated by the CGRP receptor antagonist CGRP8-37 (FIG. 1). In contrast, the linear receptor agonist Cys(Acm)2,7 that acts one of on the related amylin receptors was ineffective (FIG. 1).

### Langerin Expression and HIV-1 Degradation In Vitro

### MDLC or MULC Preparation

MDLC (1×105) and MULC (2.5×104), prepared and maintained as described (Ganor et al., Mucosal Immunol 3:506-522; Zhou, et al.; PLoS Pathog 7:e1002100.), were pretreated with CGRP 100 nM, 24 h (Sigma, St. Louis, Mo., USA), washed, and pulsed with HIV-1 JRCSF (NIH) for 2 h (10 ng corresponding to 1000 TCID50). The cells were then lysed with NP40 for HIV-1 degradation measurement by p24 ELISA (Innotest; Innogenetics, Gent, Belgium).

### Langerin Surface Expression

Flow cytometry for langerin surface expression was performed with a PE-conjugated mouse-anti-human langerin mAb (Beckman Coulter, Marseille, France). Each step was performed for 30 min on ice at 500/well.

### Results

CGRP-pretreatment resulted in a decrease in the total HIV-1 content, which correlated linearly with an increase in langerin expression (FIG. 2). Together, these results suggest that CGRP induces HIV-1 clearance and degradation by redirecting langerin to the cell surface.

### LC-TC Conjugate Formation and Mucosal TC Infection

### Inner Foreskin Explants Preparation

Inner foreskin explants were prepared, inoculated with HIV-1-infected PBMCs and processed for flow cytometry, as described (Zhou, et al.; PLoS Pathog 7:e1002100.). Briefly, inner foreskin explants were either left untreated (no CGRP) or CGRP-pretreated (100 nM CGRP) for 24 h and exposed for 4 h to either non-infected or HIV-1-infected cells.

### Results

Exposure of inner foreskin explants to HIV-1-infected cells resulted in increased LC-TC conjugate formation compared to non-infected cells (FIG. 3, mean±s.e.m fold percentage LC-TC conjugates from n=3 experiments of 2.1±0.1, p=0.0019; Student's t-test). Moreover, a small but significant proportion of TCs was infected in these explants (FIG. 3, mean±s.e.m fold percentage CD3+p24+ cells from n=3 experiments of 1.4±0.1, p=0.0293; Student's t-test). In contrast, CGRP-pretreatment abrogated both the increase in LC-TC conjugate formation and TCs infection, mediated by HIV-1-infected cells, which remained similar to that in explants exposed to non-infected cells (FIG. 3).

CGRP-pretreatment also increased langerin expression on inner foreskin epidermal Langerhans cells (mean fold±s.e.m from n=3 explants of 1.27±0.04, p=0.0280; Student's t-test). Of note, the effective concentration of 100 nM CGRP used herein in foreskin explants is within its normal range in the skin (e.g. 23 pmol CGRP per gr of skin (Ahmed et al., Neurosci Lett, 246:149-152), which translate to 65 nM CGRP, considering that normal human skin contains around 35% water). Together, these results show that both in-vitro and ex-vivo, CGRP restricts TC infection by increasing langerin expression and decreasing LC-TC conjugate formation.

### Effect of CGRP on CCL3/MIP-1α Secretion

Measure of CCL3/MIP-1α levels in culture supernatants of CGRP-treated vs. untreated MDLC and MULCs, that were pulsed with HIV-1 and co-cultured for a week with TCs were measured using a multiplex assay and flow cytometry (Bender MedSystems, Vienna, Austria) (no BMS813FF human chemokine 6plex).

### Evaluation of Transfer Inhibition

Normal goat IgG, neutralizing CCL3/MIP-1α goat Ab (R&D systems, Minneapolis, Minn., USA), or the NFκB inhibitor BAY117082 (Santa Cruz Biotechnology, Santa Cruz, Calif., USA), were included during the co-culture period (MDLC-TC) described previously. HIV-1 replication was measured in the co-culture supernatants a week later by p24 ELISA (Innotest; Innogenetics, Gent, Belgium).

### Results

The measure CCL3/MIP-1α levels in culture supernatants of CGRP-treated vs. untreated MULCs, that were pulsed with HIV-1 and co-cultured for a week with TCs, revealed that pretreatment with 100 nM CGRP resulted in increased CCL3/MIP-1α secretion (mean±s.e.m folds secretion from n=4 experiments of 1.69±0.1 (p=0.0003; Student's t-test), 0.9±0.2 and 1.2±0.2, respectively). Furthermore, the addition of CCL3/MIP-1α neutralizing Ab abrogated CGRP-mediated HIV-1 transfer inhibition from MULC to TCs in a dose-dependent manner, while a control Ab had no effect (FIG. 3A).

Further, the pharmacological NFκB inhibitor BAY117082 abrogated the inhibition of HIV-1 transfer in a dose-dependent manner (FIG. 3B). This abrogation was accompanied by a reduction in CGRP-induced elevation in CCL3/MIP-1α secretion that returned to baseline levels (mean±s.e.m fold secretion from n=4 experiments of 1.0±0.1 for the highest concentration of BAY117082).

Hence, CGRP-treatment of LCs activates NFκB, resulting in elevated secretion of the CCR5-binding chemokine CCL3/MIP-1α.

### CGRP Plasma Levels as a Biomarker to Diagnose HIV Infections and the Effectiveness of HAART

### Study Population

A total of 38 plasma samples were selected for the study, as follows: i) 14 samples from healthy individuals not exposed to HIV-1; ii) 14 samples from treatment-naive HIV-1-infected individuals, including seven primary and seven chronic HIV-1 infected patients with mean and range of CD4+ TC count of 330 (149-495)/μl and HIV-1 RNA 1154073 (5169-11000000) copies/ml. Primary HIV infection was defined on the basis of the presence of an acute clinical syndrome, a positive test for HIV-1 RNA in plasma, and the presence of less than three positive bands in a western blot assay; iii) 10 samples from HAART-treated HIV-1-infected patients with mean and range of CD4+ TC count of 738 (317-2184)/μl, on antiretroviral treatment for at least 24 and not more than 30 months with chronic and progressive infection, but without previous AIDS defining disease. Samples were matched for sex, age and risk factors. All human participants were recruited at the Department of Infectious Diseases of the San Raffaele Scientific Institute or at the Infectious Disease Clinic of the University of Milan at L. Sacco Hospital.

CGRP levels were measured in female macaques that were vaginally challenged with SHIV to mimic viral transmission during sexual intercourse as described in Bomsel et al. (Immunity, 34:269-280, 2011).

### Measurement of Plasma CGRP

An Enzyme ImmunoAssay (EIA; Bachem) was used to measure human CGRP plasma levels. For comparing human CGRP plasma levels, pair-wise comparisons were performed by the non-parametric Mann-Whitney test.

A simian ELISA (Cusabio Biotech, Wuhan, China) was used to measure CGRP plasma levels in six female Macaca mulatta placebo-vaccinated animals, before and at days 46, 72, 100, 128 (e.g. all animals infected) following repeated SHIV challenge, as described in Bomsel et al. (Immunity, 34:269-280, 2011).

### Expression of CGRP Itself and its Cognate Receptor in LCs are Up-Regulated by CGRP.

Twenty-four hours CGRP-pretreatment of MDLCs resulted in a significant increase of CGRP secretion (FIG. 8A). Moreover, it also led to an up-regulation of the two CRGP receptor subunits, CRLR and RAMP1 (FIG. 8B). This results suggest the existence of a positive loop that amplifies CRGP signaling pathway. Lower doses of CGRP could therefore be used to inhibit HIV-1 transfer from LCs to TCs.

### CGRP Applied Selectively Via the Mucosal Surface Penetrates the Epithelial Compartment of Penile Tissue.

Because HIV-1 transfer from LCs to TCs occurs within epithelia that cover mucosal surfaces of the genital tracts, the CGRP peptide penetration was evaluated in penile tissue. Polarized application to penile tissue mucosal surface selectively of Biotinylated-CGRP for 3 hrs resulted in homogeneous CGRP detection in the whole epithelial compartment while no or little HRP staining was detected in control tissue. This result reinforces the feasibility of mucosal treatment by topical application of CGRP peptides, formulated for instance as a microbicide, for the prevention of HIV transmission.

### Results

CGRP levels were significantly decreased in a group of HIV-1-infected patients compared to healthy individuals (FIG. 4). In an additional group of HIV-1-infected patients receiving highly active anti-retroviral therapy (HAART), CGRP levels were normalized back to baseline levels (FIG. 4).

CGRP plasma levels of female macaques were measured comparatively before SHIV challenge and at several later time points. This analysis showed that CGRP gradually decreased following repeated vaginal SHIV-challenges (data not shown).

Taken together, HIV-1 infection results in decreased CGRP plasma levels in both HIV-1-infected individuals and vaginally SHIV-challenged macaques.

In light of these results, CGRP is a novel HIV-1 restriction factor, which acts via its functional receptor in Langerhans cells. CGRP-treatment of Langerhans cells: i) increases cell surface expression of langerin, resulting in efficient HIV-1 clearance and degradation; ii) decreases that of selected integrins, resulting in limited formation of LC-TC conjugates; iii) activates NFκB, resulting in elevated secretion of the CCR5-binding chemokine CCL3/MIP-1α. These mechanisms cooperate to limit LC-TC viral transfer and TC infection, and contribute to the anti-HIV-1 efficacy of CGRP. This study not only reveals a novel immunosuppressive effect of CGRP on LC function and its mechanisms of action, but also provides for the first time the evidence that the nervous system can restrict ‘at a distance’ the early events of HIV-1 transmission at the mucosal level. As CGRP-containing neurons also innervate secondary lymphoid organs such as the lymph nodes, the HIV-1 inhibitory activity of CGRP may be relevant also to later time points during HIV-1 infection following LC migration and viral dissemination in such organs. Additionally, CGRP would potentially control HIV-1 spread within the nervous system itself, as CGRP exerts immunosuppressive effects also on macrophages and microglia, which are the principal cells targeted by HIV-1 in the brain. Finally, our results suggest that HIV-1 might overcome the inhibitory effect of CGRP by decreasing its mucosal levels, which are reflected in reduced CGRP levels in plasma.

Yet, based on our results, enhancing the interactions between CGRP and Langerhans cells might provide novel means for limiting HIV-1 spread, and novel CGRP receptor agonists, active especially at mucosal epithelia at targeting Langerhans cells, might represent a completely new class of anti-HIV-1 molecules.

1. CGRP and/or an agonist of the CGRP receptor for use in the prevention of an HIV infection in a human subject.

2. CGRP and/or an agonist of the CGRP receptor for use according to claim 1, wherein CGRP is the human peptide α-CGRP of sequence the sequence SEQ ID no.1 or the human peptide β-CGRP of sequence the sequence SEQ ID no.2.

3. CGRP and/or an agonist of the CGRP receptor for use according to claim 1, wherein CGRP is the human peptide α-CGRP of sequence the sequence SEQ ID no.1.

4. A method for selecting an active compound for the prevention of an HIV infection in a human subject, comprising the steps of:
a) contacting Langerhans cells with a candidate compound;
b) in vitro infecting of Langerhans cells by at least one HIV variant,
c) measuring of at least one of the following parameters:
HIV content,
adhesive potential of Langerhans cells, in particular to TCs,
secretion of anti-HIV chemokines by Langerhans cells.

5. A method according to claim 4, characterized in that it comprises the steps of:
a) contacting Langerhans cells with a candidate compound;
b) in vitro infecting of Langerhans cells from step a) by at least one HIV variant,
c) growing infected Langerhans cells from step b) with CD4+ T cells in culture medium;
d) measuring at least one of the following parameters:
HIV content
adhesive potential of Langerhans cells, in particular to TCs, or
secretion of anti-HIV chemokines by Langerhans cells.

6. A method according to claim 4 or 5, characterized in that it further comprises at least one of the following steps:
i. The HIV content is compared to a HIV content threshold;
ii. The adhesive potential of Langerhans cells is compared to an adhesive potential of Langerhans cells threshold;
iii. The secretion of anti-HIV chemokines by Langerhans cells is compared to a secretion of anti-HIV chemokines threshold.

7. A method according to claim 4 or 5, characterized in that said candidate compound is selected as an active compound for the prevention of an HIV infection in a human subject if:
i. The HIV replication content is compared to a HIV replication content threshold; and
ii. The adhesive potential of Langerhans cells is compared to an adhesive potential of Langerhans cells threshold; and
iii. The secretion of anti-HIV chemokines by Langerhans cells is compared to a secretion of anti-HIV chemokines threshold.

8. A composition comprising CGRP and/or agonist of the CGRP receptor for use in the prevention of an HIV infection in a human subject.

9. A composition according to claim 8, characterized in that it is formulated for cutaneous administration.

10. A method for the in vitro diagnosis of an HIV infection in a human subject, characterized in that it comprises determining the level of CGRP from a biological sample from said subject.

11. A method according to claim 10, characterized in that it comprises the following steps:
a) determining the level of CGRP from a biological sample from said subject; and
b) concluding to an HIV infection in said subject if the level of CGRP from step a) is inferior to the normal level of CGRP in the biological sample from non-infected human subjects.


SEQUENCE LISTING (3 sequences)

SEQ ID NO: 1 - protein, 37 aa, homo sapiens
  MOD_RES (37)..(37): AMIDATION
ACDTATCVTHRLAGLLSRSGGVVKNNFVPTNVGSKAF

SEQ ID NO: 2 - protein, 37 aa, homo sapiens
  MOD_RES (37)..(37): AMIDATION
ACNTATCVTHRLAGLLSRSGGMVKSNFVPTNVGSKAF

SEQ ID NO: 3 - protein, 7 aa, artificial
  Truncation of alpha-CGRP protein
ACDTATC